(19) United States
(12) Patent Application Publication
(10) Pub. No.: US 2009/0082692 A1
(43) Pub. Date: Mar. 26, 2009

(54) System And Method For The Real-Time Evaluation Of Time-Locked Physiological Measures

(76) Inventors: Kelly S. Hale, Oviedo, FL (US); Sven Fuchs, Westerronfeld (DE); Christine Berka, Carlsbad, CA (US)

Correspondence Address: BEUSSE WOLTER SANKS MORA & MAIRE, P. A., 390 NORTH ORANGE AVENUE, SUITE 2500, ORLANDO, FL, 32801, US

(21) Appl. No.: 12/237,985

(22) Filed: Sep. 25, 2008

Related U.S. Application Data
(60) Provisional application No. 60/974,956, filed on Sep. 25, 2007.

(51) Int. Cl. A61B 5/0476; A61B 5/00

(52) U.S. Cl. 600/544; 600/301

(57) ABSTRACT

In one embodiment, a method is provided for classifying cognitive activity in an individual. In the method, a candidate time interval is identified from a first type of physiological data within which cognitive processing is expected to occur for the individual. In addition, a second type of physiological data is obtained that comprises data representative of a cognitive state of the individual. Further, the data representative of a cognitive state of the individual is extracted from the second type of physiological data based on the identified candidate time interval.

This application claims benefit under 35 USC 119(e)(1) of the Sep. 25, 2007 filing date of U.S. Provisional Application Nos. 60/974,956, the entirety of which is incorporated by reference herein.

### STATEMENT REGARDING FEDERALLY SPONSORED DEVELOPMENT

Development for this invention was supported in part by Contract No. FA8750-06-C-0197 issues by the Air Force Research Laboratory (AFRL) and made under the Intelligence Advanced Research Projects Activity (IARPA) Collaboration and Analyst System Effectiveness (CASE) Program. Accordingly, the government may have certain rights in the invention.

### FIELD OF THE INVENTION

The present invention relates to the field of physiological measurement, and more particularly to a system and method for the real-time detection and evaluation of a cognitive state of an individual.

### BACKGROUND OF THE INVENTION

Many complex task environments, such as those in air traffic control, power plant control rooms, military command-and-control systems, or emergency response centers, require individuals to maintain situation awareness while being exposed to ever-increasing amounts of data that may obscure relevant information and exceed the natural limitations of human information processing (HIP). These cognitive overload conditions can lead to reduced performance and human error, with potentially disastrous consequences in the case of safety-critical environments.

It has been recognized that monitoring the human physiology may result in early identification of HIP-related problems and enable dynamic adaptation of the task environment to account for the human operator's cognitive state and mitigate such problems before cognitive breakdown occurs. HIP assessment may also provide benefits in a wide variety of other environments where HIP determines the probable outcome of an action. Examples of such environments are education and training, operating machinery (including driving) or entertainment (including gaming).

Currently, assessment of HIP is enabled by physiological sensors capable of identifying cognitive processing and distinguishing cognitive states in an individual. While some physiological indices are quantitative in nature, (e.g. a threshold can be applied to easily detect changes in a cognitive state; see Berka et al. (2007). EEG Correlates of Task Engagement and Mental Workload in Vigilance, Learning and Memory Tasks. Aviation Space and Environmental Medicine, 78 (5, Section II, Suppl.), other indicators are embedded in continuous data streams and require processing that is too complex to achieve continuous evaluation in real-time. Nevertheless, because real-time assessment is critical to make HIP assessment applicable to real world operational settings, scientists have attempted to develop a solution. In an attempt to obtain the desired classifiers indicating a change in cognitive state, an analysis time window must be aligned appropriately to extract a relevant portion of the data stream, which can then be analyzed in isolation. Typically, these analysis windows are aligned to the presentation of known stimuli occurring at a known time. The known stimuli are expected to trigger a change in cognitive state. In laboratory settings, where quality and onset of the stimulus are known and controlled, alignment to external events is feasible. However, in natural operational environments, there is currently no method for determining or predicting the occurrence of a relevant event around which an analysis window must be placed.

### BRIEF DESCRIPTION OF THE INVENTION

In accordance with one aspect of the present invention, there is provided a method for classifying cognitive activity in an individual. In the method, one or more candidate time intervals are identified from a first type of physiological data within which cognitive processing is expected to occur for the individual. In addition, a second type of physiological data is obtained that comprises data representative of a cognitive state of the individual. Further, the method includes extracting data representative of a cognitive state of the individual from the second type of physiological data based on the identified candidate time interval.

In accordance with another aspect of the present invention, there is provided a system for classifying cognitive activity in an individual. The system comprises a first sensor configured to acquire a first type of physiological data from the individual and a second sensor configured to acquire a second type of physiological data from the individual. The second type of physiological data comprises data representative of a cognitive state of the individual. The system further comprises a processor coupled to the first sensor and the second sensor and configured to: (a) identify a candidate time interval in the first type of physiological data within which cognitive processing is expected to occur for the individual; (b) extract the data representative of a cognitive state of the individual from the second type of physiological data based on the candidate time interval; and (c) identify the cognitive state of the individual by comparing the extracted data to known standards stored in a memory.

### BRIEF DESCRIPTION OF THE DRAWINGS

The invention is explained in the following description in view of the drawings that show:

FIG. 1 is a schematic diagram showing an embodiment of a system according to an aspect of the present invention;

FIG. 2 is a flow diagram showing the operation of a system according to an aspect of the present invention;

FIG. 3 is a graph showing three different time intervals placed on a Type-B sensor data stream in accordance with the present invention;

FIG. 4 is a schematic of a data system in communication with two sensors in accordance with the present invention;

FIG. 5 is a flow diagram showing a closed loop system in accordance with an aspect of the present invention; and

FIG. 6 is a flow diagram of a method according to an aspect of the present invention.

### DETAILED DESCRIPTION OF THE INVENTION

In accordance with particular aspects of the present invention, the inventors of the present invention have developed a method and system for the real-time or near real-time assessment of cognitive activity in an individual that predicts the occurrence of a relevant event around which an analysis window may be placed. Aspects of the present invention thus eliminate the need to know or define a-priori the temporal parameters of an event to which the individual's physiological reaction is time-locked. Further, the analysis of an event-locked physiological signal (used to classify a cognitive state) is initiated by a time-sensitive indicator of cognitive processing that originates from the individual himself.

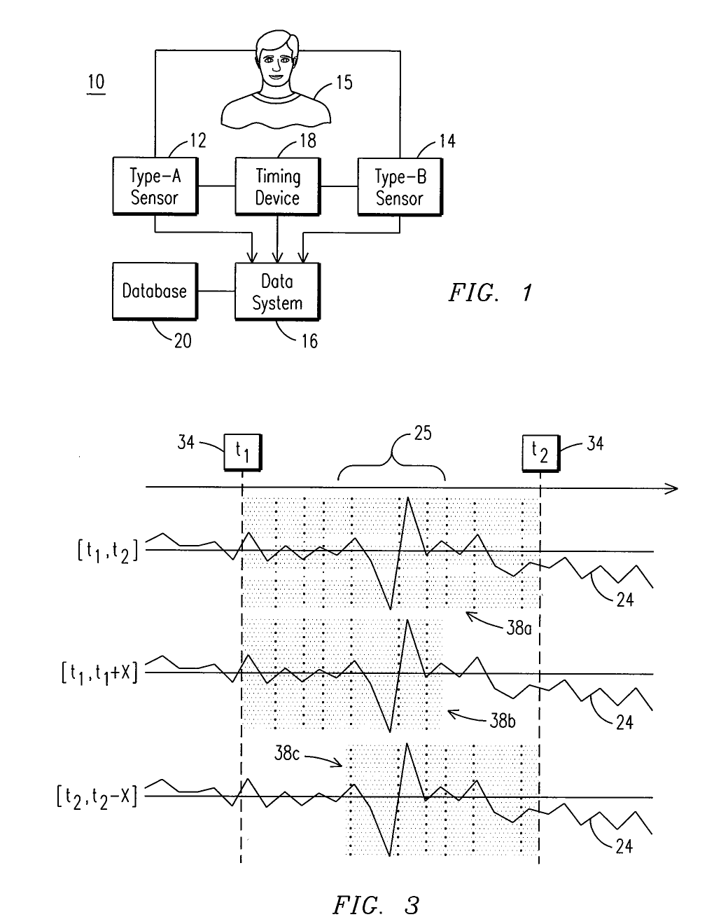

Now referring to the drawings, FIG. 1 depicts a system 10 for classifying cognitive activity in an individual that eliminates the need for predicted external stimuli to access the cognitive activity of an individual. The system 10 comprises a first physiological sensor (hereinafter Type A sensor 12), a second physiological sensor (hereinafter a Type-B sensor 14), a data system 16, a timing device 18, and a database 20. It is understood that the timing device and database 20 may be a part of the data system 16, but are shown separately for convenience. The sensors 12,14 are associated with the individual 15 via direct or indirect contact via any suitable method known in the art. The Type-A sensor 12 may be any suitable physiological sensor that provides physiological data capable of being analyzed in real-time or near real-time. As will be discussed below, the data from the Type-A sensor 12 may be utilized to identify a time interval within which cognitive activity (also referred to as cognitive processing herein) is expected to be taking place.

In an embodiment, the Type-A sensor 12 provides data representing an expected time frame (candidate time interval) wherein cognitive activity takes place in response to an event. By “individual,” as used herein, it is meant any human being or animal. By “an event,” it is meant the occurrence of a cognitive reaction to any stimulus or activity, such as an audio or visual stimulus, texts, writings, photographs, and the like, that is capable of causing cognitive activity in an individual. In an embodiment, the timing and/or occurrence of the event is not known to the individual or other person prior to the event's occurrence, but is instead a random occurrence or an event that is relatively certain to occur at a point in time, but the time of which is uncertain. For example, an individual may read text and recognize a relevant portion. This recognition would not be a ‘known event’ because the system does not know what portion of the text (if any) is relevant or recognizable to the individual.

In an embodiment, the Type-A sensor 12 comprises one or more eye tracking devices, e.g. an electrooculograph, capable of obtaining data from the eye activity of an individual. The Type-A sensor is capable of determining a point in time or time window (hereinafter a candidate time interval) associated with expected cognitive activity. For example, an eye sensor for detecting and measuring the duration of ocular fixations may be used for the purpose of identifying a candidate time interval based on a duration of an ocular fixation. An ocular fixation may be defined as a group of successive gaze points in which the location of one gaze point occurs within a certain time (e.g., 20 msec) and distance (e.g., 50 pixels) of the previous gaze point. The fixation duration may be defined as the candidate time interval from the onset of the first gaze point in the group to the offset of the last gaze point of the group. It is believed that the duration of ocular fixations is longer when a stimulus, e.g. a visual stimulus attracts the attention of the individual, thereby indicating cognitive activity (i.e., focused attention).

Alternatively, the Type-A sensor 12 may be any other physiological sensor for obtaining information useful for defining a candidate time interval that is identified with cognitive activity, including other known processes for defining ocular fixations and their duration. Alternative embodiments may include one or more Type-A physiological sensors 12 and associated algorithm(s) to derive quantitative measures, including but not limited to the size of the pupil, which fluctuates in milliseconds based on cognitive activity of individual, the position of ocular fixations, and the time of a relevant change in electrocardiogram (ECG), galvanic skin response, or skin temperature.

In embodiments of the present invention, as will be discussed below, the candidate time interval derived from the output of the Type-A sensor 12 may be used for the definition of a candidate interval within which one or more cognitive state indicators (as will be explained below) is expected to occur in the data stream of the Type-B sensor 14. The Type-B sensor 14 may be any suitable sensor for producing a data stream comprising a cognitive state indicator. In an embodiment, the recognition of the cognitive state indicator requires: (a) the definition of a candidate time interval derived from the Type-A sensor 12 within the stream that includes the embedded cognitive state indicator; and (b) the isolated processing and analysis of data from the Type-B sensor 14 contained within the candidate time interval (or within a time interval slightly adjusted from the candidate time interval). The cognitive state indicator may comprise any data set that is operative to identify or indicate a cognitive state of an individual, such as attention, perception, comprehension, situation awareness, recognition, cognitive workload, alertness, engagement, drowsiness, bias, or confusion, and the like. As will be discussed below, the cognitive state indicator may also be used to provide a hierarchy of probable cognitive states for the individual from the data by comparing the data set to known data sets (known standards) representing a particular cognitive state. In other words, the cognitive state indicator may identify a particular classification in order of probability from a plurality of possible classifications.

In an embodiment, the Type-B sensor 14 comprises one or more electroencephalograph (EEG) sensors, each of which are configured to acquire EEG signals from a plurality of locations on the individual 15, e.g. the head, to provide a continuous data stream having at least one cognitive state classifier embedded therein. By “embedded,” it is meant that some processing of the data is necessary to interpret its meaning. For applications in operational or other real-world environments, the number of EEG sensors should be kept at a minimum (three to twenty) to allow portability, minimize power consumption and maintain comfort and ease of use. Cognitive states, such as attention, alertness, and mental workload may be characterized using a five-sensor array. Several documented event-related neural signatures (e.g. P300 or Late Positive Component) are sufficiently robust to be detectable using only one or two sensor placements with EEG. However, larger sensor arrays are recommended during the stage where identification and characterization of novel signatures is undertaken. It is anticipated that, in alternative embodiments, the EEG signal may be combined or replaced with recorded data obtained from other sensors that deliver additional physiological signals, such as the electromyogram (EMG), electrocardiogram (ECG), functional near infrared spectroscopy (fNIR), respiratory activity, head or body movement, or galvanic skin response (GSR), or skin temperature.

The data system 16 typically comprises one or more computing devices, each typically having inputs, a processor, and a memory (not shown) to monitor the data provided by the Type-A sensor 12 and the Type-B sensor 14. The sensors 12, 14 periodically or continuously acquire data from the individual 15 to produce a data stream 22 from the Type-A sensor 12 and a data stream 24 from the Type-B sensor 14. The data streams 22,24 may be directed to one or more computing devices of the data system 16. In this way, the data system 16 is in communication with the Type-A sensor 12 to store the data generated by the Type-A sensor 12 in a memory (or alternatively in an external memory device). In the same way, the data system 16 is in communication with the Type-B sensor 14 to store data generated by the Type-B sensor 14 in the memory (or alternatively in an external memory device).

The data from the Type-A sensor 12 or Type-B sensor 14, particularly the Type-A data, may be continuously analyzed in real-time or near real-time by the data system 16 for evidence of expected cognitive activity. In an embodiment, an indication of expected cognitive activity in the individual is continuously analyzed by monitoring and interpreting fluctuations of quantitative metrics, which can be obtained from experimentation or other means of cognitive assessment as are well-known in the art. However, those skilled in the art will also appreciate that indicators for cognitive activity, such as a change in cognitive state of the individual, may not necessarily be comprised of a cognitive activity quantitative metric, instead the change in cognitive state may be measured quantitatively or qualitatively by facial expression, voice intonation, or postural control.

In an embodiment, the data system 16 further includes software, hardware, or the like for analyzing, managing, and/or processing the data from the Type-A sensor 12 and/or the Type-B sensor 14 to be implemented by the processor of the data system 16. For example, in an embodiment, the data system 16 may include software to process the time-locked data from the Type-B sensor 14 before the data from the Type-B sensor 14 is compared to known standards or templates. In addition, the data system 16 may include software or for synchronizing the data from the sensors 12, 14 as will be discussed below, such as an External Synchronization Unit. Further, the data system 16 may further include software for classifying the data from the sensors 12, 14 by comparing the captured data to known standards, such as templates of event-related potentials that indicate cognitive activity in EEG data.

One or more timing devices 18 (hereinafter timing device 18) may be in electrical communication with the Type-A sensor 12, the Type-B sensor 14, and/or the data system 16 to generate or aid a processor of a computing device within the data system 16 in generating one or timestamps representing one or more endpoints of an interval of expected cognitive activity for the individual. The timing device 18 may thus be included as software on a computing device of the data system 16 or may be a peripheral device in communication with a computing device of the data system 16.

One or more databases (hereinafter database 20) may also be in communication with or provided as part of the data system 16 or may be provided as a separate peripheral device or on a suitable memory storage device. In an embodiment, the database 20 includes at least one, and typically a plurality of known data sets (known standards or templates) representing a particular cognitive state, e.g. attention, recognition, cognitive processing, cognitive overload, alertness, mental workload, bias, confusion, and the like. The templates are a subset of features or combinations of features extracted from either the signals of Type-B sensors or a combination of signals from both A and B sensor types that optimize discriminability between classes of cognitive states. In an embodiment, the database of cognitive state templates is obtained using experimental conditions which elicit the targeted state(s) or control environmental or psychological factors to better isolate an intended state(s). One skilled in the art may design any number of experiments that could be used to derive templates for the identification of one or more cognitive states, which may include but are not limited to templates for the evaluation of signal detection (e.g. hits, misses, false alarms, correct rejections), decision making and comprehension, as well as the recognition of interest, errors and cognitive biases, and the like. In a particular embodiment, the database 20 comprises a plurality of known patterns of EEG data that correspond to a particular cognitive state. Accordingly, when an unknown data set of EEG data (or other data type) is compared with templates of the same data type, a probability distribution may be provided that sets forth the likelihood that the unknown data set from the Type-B sensor 14 corresponds to a particular cognitive state.

In an embodiment, the transmission of the physiological signals between sensors 12, 14, which may be mounted on the individual 15, and the data system 16 that performs the analysis and assessment of cognitive states, is provided wirelessly. However, those skilled in the art recognize that wireless transmission of either analog or digital physiological signals enables the user to be more mobile during use, but may also increase the signal to noise ratio. In another embodiment, the transmission of physiological signals between the sensors 12, 14 and the data system 20 may be done by wired methods using optics, cables, harnesses, or the like.

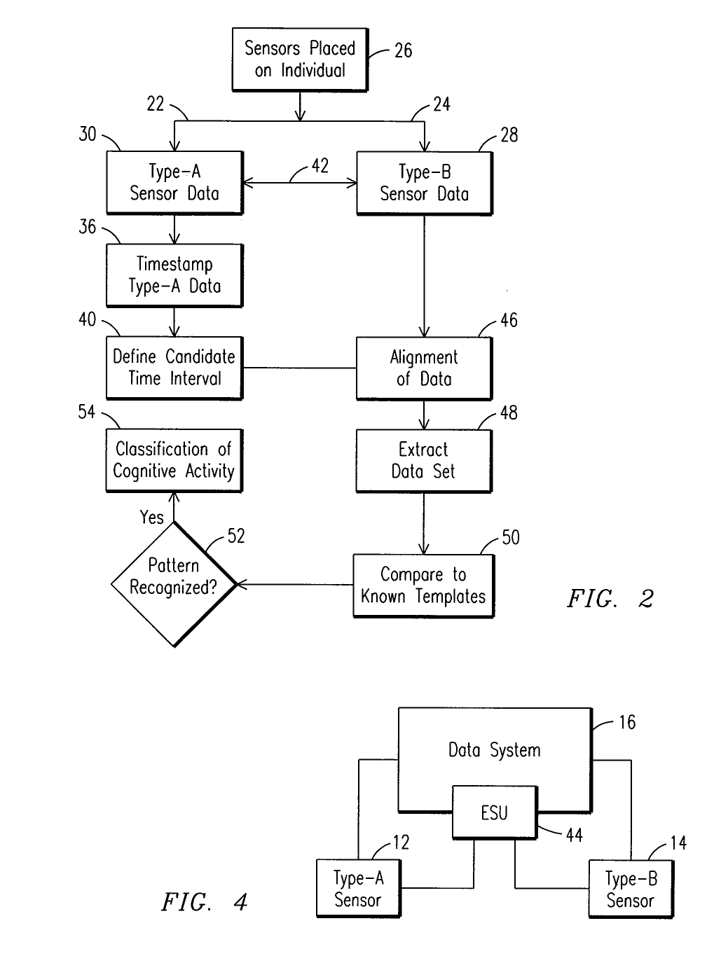

Now referring to FIG. 2, there is shown and described below a flowchart of an embodiment of the system 10 in operation for determining and/or classifying the cognitive activity of an individual 15. First, at reference numeral 26, a facilitator (not shown) positions the physiological sensors in an adequate position with regard to the individual 15 as is known in the art. The physiological sensors include at least one Type-A sensor 12 and one Type-B sensor 14. One sensor of each type is described, but it is understood the invention is not so limited. At reference numerals 28 and 30, upon commencement of monitoring the individual, both sensors 12, 14 periodically or continuously acquire data from the individual 15 and direct the corresponding data streams 22, 24 may be directed to one or more computing devices of the data system 16.

When the cognitive activity of the individual 15 is found to be consistent with expected cognitive activity, such as in response to an event as described above, the data from the Type-A sensor is provided with one or more timestamps 34 associated with the expected timeframe of cognitive activity at reference numeral 36. The timestamps 34 (shown in FIG. 3) may be provided, for example, when the data system 16 detects a change in a quantitative measure or measurements above a certain threshold. Thereafter, the timestamps 34 may be utilized to define a candidate time interval, e.g. time intervals 38a-c, from the data stream 22 from the Type-A sensor 12 at reference numeral 40. The candidate time intervals 38a-c shown may be a time period between a first timestamp and a second timestamp on the Type-A data stream 22, a period before a timestamp, or a period after a timestamp. In an embodiment, the candidate time interval is identified by the sensor 12 and/or a processor of the data system 16 as a result of the individual's response to a spontaneous event in real-time or near real-time.

Although an embodiment of a single time interval having two endpoints is described herein, it is understood that one or more time intervals may be provided, each of which indicate a timeframe where cognitive activity 25 (also referred to as cognitive processing) is expected to have taken place. Further, it is understood that the time interval may include only a single endpoint and that the corresponding Type-B sensor data may be extracted for a certain period before or after that endpoint.

Referring to FIG. 3, for example, there is shown a first time interval 38a defined between two endpoints t₁ and t₂. Two endpoints may be provided to define the candidate time interval when, for example, an individual suddenly gazes upon a particular object, but later turns away. In another embodiment, there is shown a second time interval 38b defined by an initial endpoint (t₁) and an additional length of time thereafter (x). In this case, the second time interval 38b (t₁, t₁+x) may be utilized for example when an individual first hears an auditory signal. In such a case, one may want to review the individual's cognitive activity for a predefined time thereafter to define a particular cognitive state for such time, e.g. whether the signal was correctly interpreted. Further, in another embodiment, there is shown a third time interval 38c defined by an endpoint t₂ and a time period prior to the endpoint (x). In this case, the third time interval 38c (t₂, t₂−x) may be utilized when an individual makes a sudden movement, e.g. a run for shelter, and one may desire to evaluate the cognitive activity that caused the event, e.g. recognition of a threat prior to the running action.

The candidate time interval derived from the output of the Type-A sensor 12 is used for the definition of a time interval within which one or more cognitive state indicators is expected to occur in the data stream of the Type-B sensor 14. To increase the likelihood that a cognitive state indicator will be found in the candidate time interval 38 (identified by the Type-A sensor data) in the Type-B data stream, those skilled in the art will appreciate that synchronization and alignment of the Type-A sensor and Type-B sensor data may be necessary.

In order for the physiological state to be accurately classified, the signals obtained from each of the Type-A sensor 12 and the Type-B sensor 14 may further be at least temporally synchronized as indicated by arrow 42. Any significant delay (e.g., greater than 25 ms) in the integration of sensor signals into a data reduction and analysis routine of the data system 16, for example, may impact the accuracy of the cognitive state classifier (data indicating a particular cognitive state), particularly in a system designed to detect a plethora of cognitive states.

In a particular embodiment, as shown in FIG. 4, the data system 16 may comprise an External Synchronization Unit (ESU) 44 that is designed to synchronize upon receipt or input of the data from the physiological sensors (Type-A sensor 12 and Type-B sensor 14) and/or any other system that the user is interacting with (e.g., a software platform). In addition, the ESU 44 may provide a common timestamp 34 to allow synchronization across inputs with precision at the millisecond level. In alternative embodiments, a Unix, Linux, or other operating system or machine language application that provides control over the sensors 12, 14 and optionally the data system 16 may be used to perform the necessary synchronization. In an alternate embodiment, the data system 16 may include a plurality of computing devices and a plurality of timing devices 18 to synchronize multiple computing devices in order to acquire sensor data from sensors 12, 14 and/or to provide environmental triggers or events.

Further, at reference numeral 46, the Type A-sensor and the Type-B sensor data streams 22,24 may optionally be aligned such that the candidate time interval 38 may be positioned on the Type-B sensor data stream 24. One skilled in the art will recognize that, depending on the cognitive activity to be evaluated, several ways of alignment are possible. Some event-related changes may occur simultaneously with the detected cognitive activity. Other event-related changes in physiological signals acquired by a Type-B sensor 14 may occur subsequent to or prior to the cognitive activity detected by a Type-A sensor 12. The exact length and position of the time interval typically depends on the cognitive state to be assessed, the sensors used, and the classifiers employed in the analysis of the data. Hence, the present invention recognizes that the candidate time interval derived from the Type-A data may be located before, on, or after the generated time stamp, which may require Type-B signal samples to be stored in and retrieved from a memory. The data system 16 may include a dedicated memory space for this purpose. For this reason, the actual time interval placed on the Type-B data may be said to be “based upon” or “based on” the candidate time interval as the actual time interval may be identical or slightly adjusted in either direction. One skilled in the art would also appreciate that alignment of the data may not be necessary if the Type-B data is of such a quality that it does not require Type-A data to define a time window (interval). Generally, however, alignment will be required.

Thereafter, as shown at reference numeral 48, once one or more time intervals, e.g. one of intervals 38a-c, has been identified within which cognitive activity 25 is expected to have occurred, the portions of the Type-B data stream 24 corresponding to the candidate time interval (as adjusted if necessary) may be extracted from the continuous data stream provided by the Type-B sensor 14 to provide one or more extracted data sets. At reference numeral 50, the extracted data set may be compared to known pattern templates provided in the one or more databases, e.g. database 20, of the data system 16. If the pattern is recognized as indicated by reference number 52, the pattern may be classified as shown by reference numeral 54. In this way, each cognitive state identified by the Type-B data may be recognized by comparing the elementary patterns of the resulting data to one or more pattern templates of known cognitive states. The ability to detect particular cognitive states will depend on the content of the database, e.g. database 20, of templates specific to, or predictive of, cognitive states, which were previously obtained using experimental methods or derived from published sources. A given cognitive state is recognized and classified if the elementary features satisfy a set of criteria associated with the template for that cognitive state. As discussed previously, the portion of the Type-B signal that falls within the candidate time interval may be first processed by applying an adequate combination of data processing methods associated with the templates prior to the attempted classification of the Type-B data falling within the particular window.

In an embodiment, the recognition and classification of a specified cognitive state is obtained from a classification algorithm that compares the Type-B signal in the analysis window to existing cognitive state templates. In an embodiment, stepwise regression analysis may be employed to select features that best discriminate classes of events (e.g. hit, misses) and then linear discriminant function analysis may be employed to provide event classification. In other embodiments, a variety of real-time classification techniques could be deployed, such as logistic regression analysis, K Nearest Neighbor, Parzen Windows, Gaussian Mixture Models, fuzzy logic classifiers and/or Artificial Neural Networks. Cognitive state recognition could be a simple bi-modal approach (e.g., correct vs. incorrect) or a multi-layered approach which utilizes multiple cognitive state algorithms.

The herein described methods and systems for analyzing and/or classifying event-evoked cognitive states for an individual can be applied in real-time or near real-time, independent of the user environment or event condition, assuming that adequate computing equipment with sufficient processing power is used for near-real-time analysis of the data streams. Aspects of the present invention are particularly beneficial in non-deterministic environments where it is not known whether and when an event will occur such that the associated physiological signals may be used to indicate the occurrence of an unknown or undetectable event or cognitive processing associated with the unknown or undetectable event. The utility of the described method is wide-spread as it can be used to assess a plurality of cognitive states by way of time-synchronized physiological sensors. The range of possible evaluations is dependent on the available indicators and pattern templates for cognitive state assessment.

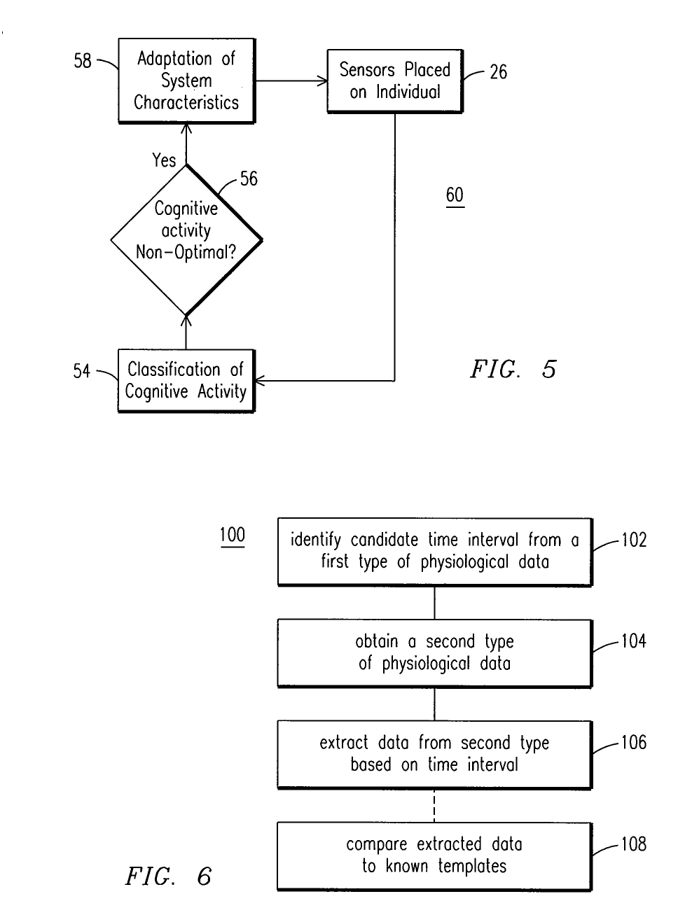

In an embodiment, as is further shown in FIG. 5, the described system 10 is an interactive system where the successful cognitive state classification at reference numeral 54 may be used to create a closed-loop 60 involving real-time modification of system characteristics in a way that the cognitive state of the individual 15 is accounted for. As shown, at reference numeral 56, a non-optimal cognitive state is detected. In response, the system 10 may adapt in a way that alleviates the problematic cognitive state to provide the closed loop system 60. In particular, the closed loop 60 is created when an adaptation is provided at reference numeral 58 that has an effect on the cognitive state of the individual. Upon implementation of the adaptation, the system and process shown in FIG. 2 may be completed again and again (if necessary) until the non-optimal cognitive state is no longer present in the individual 15. For convenience, the system and process shown in FIG. 2 is not shown again, but the arrow from 26 to 54 is understood to include all the elements shown in FIG. 2. In an embodiment, the adaptation may address the presentation of information. For example, the system 10 may evaluate target-related decision making in a target detection task, such as the analysis of geospatial data to detect enemy units or the location of Improvised Explosive Devices (IEDs). If signature data from the Type-B sensor 14 (e.g. ERP data) associated with prolonged ocular fixations derived from a Type-A sensor do not indicate proper decision making, the image or a portion of an image (for example) may be repeatedly displayed until a proper decision is detected. Other embodiments that benefit from event-evoked cognitive state assessment include, but are not limited to, the evaluation of performance, the optimization of operator's aftentional focus, or the mitigation of cognitive biases.

In accordance with another aspect of the present invention, there is provided a method 100 for utilizing the above-described system. The method comprises step 102 of identifying a candidate time interval from a first type of physiological data within which cognitive processing is expected to occur for an individual 15. In addition, the method comprises step 104 of obtaining a second type of physiological data comprising data representative of a cognitive state of the individual 15. Further, the method comprises step 106 of extracting the data representative of a cognitive state of the individual from the second type of physiological data based on the identified candidate time interval 38. In an embodiment, the method comprises the additional step 108 of identifying the cognitive state of the individual by comparing the extracted data to known standards representing a particular cognitive state.

In an embodiment, step 102 is performed via the first sensor (Type-A sensor 12) and a processor, which is typically part of the data system 16. In this embodiment, the first sensor may be an eye tracking sensor configured to obtain eye activity from the individual and the processor is configured to determine a candidate time interval within which eye activity occurs. In a particular embodiment, the processor determines the candidate time interval by a duration of an ocular fixation. In addition, the second type of physiological data (from the Type-B sensor) may be in the form of a continuous data stream and the data representative of a cognitive state of the individual may be embedded in the continuous data stream. The embedded data can be obtained as previously described and compared to known standards.

It is understood when an element as described herein is used in the singular form, e.g. “a” or as “one or more,” or the like, the element is not so limited to the singular form, but may also encompass a plurality of such elements.

Based on the foregoing specification, the above-discussed embodiments of the invention may be implemented using computer programming or engineering techniques including computer software, firmware, hardware or any combination or subset thereof, wherein the technical effect is to analyze, manage, and/or process the data from the Type-A sensor 12, the Type-B sensor 14, the data system 16, or other any component and compare experimental data to known data, as well as carry out the other tasks described herein. Any such resulting program, having computer-readable code means, may be embodied or provided within one or more computer-readable media, thereby making a computer program product, i.e., an article of manufacture, according to the discussed embodiments of the invention. The computer readable media may be, for instance, a fixed (hard) drive, diskette, optical disk, magnetic tape, semiconductor memory such as read-only memory (ROM), etc., or any transmitting/receiving medium such as the Internet or other communication network or link. The article of manufacture containing the computer code may be made and/or used by executing the code directly from one medium, by copying the code from one medium to another medium, or by transmitting the code over a network.

One skilled in the art of computer science will easily be able to combine the software created as described with appropriate general purpose or special purpose computer hardware, such as a microprocessor, to create a computer system or computer sub-system of the method embodiment of the invention. An apparatus for making, using or selling embodiments of the invention may be one or more processing systems including, but not limited to, a central processing unit (CPU), memory, storage devices, communication links and devices, servers, I/O devices, or any sub-components of one or more processing systems, including software, firmware, hardware or any combination or subset thereof, which embody those discussed embodiments the invention.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The invention claimed is:

1. A method for classifying cognitive activity in an individual comprising:
(a) identifying a candidate time interval from a first type of physiological data within which cognitive processing is expected to occur for the individual;
(b) obtaining a second type of physiological data comprising data representative of a cognitive state of the individual; and
(c) extracting the data representative of a cognitive state of the individual from the second type of physiological data based on the identified candidate time interval.

2. The method of claim 1, further comprising step (d) of identifying the cognitive state of the individual by comparing the extracted data to known standards representing a particular cognitive state.

3. The method of claim 2, wherein the cognitive processing identified by said step (a) of identifying is evoked by an event, and wherein the method further comprises representing the event to the individual after said step (d) of identifying until a change in the cognitive state is identified.

4. The method of claim 1, wherein the cognitive state is at least one of attention, perception, comprehension, situation awareness, recognition, cognitive workload, alertness, engagement, drowsiness, bias, or confusion.

5. The method of claim 1, wherein the candidate time interval identified by said step (a) of identifying is evoked by a spontaneous event in real-time or near real-time.

6. The method of claim 1, wherein the first type of physiological data and the second type of physiological data are obtained via a first sensor and a second sensor respectively, and wherein the method further comprises synchronizing an output of the first sensor and the second sensor.

7. The method of claim 1, wherein said (a) of identifying is done via a first sensor and a processor, wherein the first sensor is an eye tracking sensor configured to obtain eye activity from the individual, and wherein the processor is configured to determine the candidate time interval within which eye activity occurs.

8. The method of claim 7, wherein the processor determines the candidate time interval by a duration of an ocular fixation.

9. The method of claim 1, wherein the candidate time interval comprises a first endpoint and a second endpoint.

10. The method of claim 1, wherein the candidate time interval comprises a period of time before or after an endpoint.

11. The method of claim 1, wherein the second type of physiological data is in the form of a continuous data stream, and wherein the data representative of a cognitive state of the individual is embedded in the continuous data stream.

12. The method of claim 1, wherein said step (b) of obtaining is done via a second sensor, and wherein the second sensor is an EEG sensor.

13. A system for classifying cognitive activity in an individual comprising:
a first sensor configured to acquire a first type of physiological data from the individual;
a second sensor configured to acquire a second type of physiological data from the individual, wherein the second type of physiological data comprises data representative of a cognitive state of the individual; and
a processor coupled to the first sensor and the second sensor and configured to:
identify a candidate time interval in the first type of physiological data within which cognitive processing is expected to occur for the individual;
extract the data representative of a cognitive state of the individual from the second type of physiological data based on the candidate time interval; and
identify the cognitive state of the individual by comparing the extracted data to at least one standard stored in a memory.

14. The system of claim 13, wherein the cognitive state is at least one of attention, perception, comprehension, situation awareness, recognition, cognitive workload, alertness, engagement, drowsiness, bias, or confusion.

15. The system of claim 13, wherein the cognitive processing is event-evoked cognitive processing.

16. The system of claim 13, wherein the processor is further configured to represent an event to the individual until a change in the cognitive state is identified.

17. The system of claim 13, wherein the processor is further configured to synchronize an output of the first sensor and the second sensor.

18. The system of claim 13, wherein the first sensor is an eye tracking sensor configured to obtain eye activity from the individual, and wherein the processor is configured to determine the candidate time interval from the eye activity.

19. The system of claim 18, wherein the processor is further configured to determine the time interval by a duration of an ocular fixation.

20. The system of claim 13, wherein the second type of physiological data is in the form of a continuous data stream, and wherein the data representative of a cognitive state of the individual is embedded in the continuous data stream.

21. The system of claim 13, wherein the second sensor is an EEG sensor.

22. The system of claim 13, wherein the time interval comprises a first endpoint and a second endpoint.

23. The system of claim 13, wherein the processor is configured to process the data from the first sensor and the second sensor in real-time.
